(19) United States
(12) Patent Application Publication
(10) Pub. No.: US 2011/0112423 A1
(43) Pub. Date: May 12, 2011

(54) INCREASING CPR EFFECTIVENESS USING PHONOCARDIOGRAM ANALYSIS

(75) Inventors: Fred Chapman, Newcastle, WA (US); Joseph L. Sullivan, Kirkland, WA (US); Mitchell A. Smith, Sammamish, WA (US)

(73) Assignee: PHYSIO-CONTROL, INC., Redmond, WA (US)

(21) Appl. No.: 12/833,724

(22) Filed: Jul. 9, 2010

Related U.S. Application Data
(60) Provisional application No. 61/260,308, filed on Nov. 11, 2009.

(51) Int. Cl. A61B 5/02

(52) U.S. Cl. 600/528

(57) ABSTRACT

Embodiments of the invention include a system and methods for providing status information about resuscitation efforts of a person receiving chest compressions as part of Cardiopulmonary Resuscitation (CPR). A microphone generates a soundtrack by sampling sounds within or around the body of the person receiving CPR. The soundtrack is gated in one or more various ways to eliminate portions of the soundtrack, and analysis performed on the remaining portions. By evaluating the remaining portions of the soundtrack, the system can determine a cardiovascular effect of the compressions and provide status information to the rescuer about the determined cardiovascular effect.

### CROSS REFERENCE TO RELATED PATENT APPLICATIONS

This patent application claims priority from U.S. Provisional Patent Application Ser. No. 61/260,308, filed on Nov. 11, 2009, entitled USING PCG TO DETERMINE CPR EFFECTIVENESS, the disclosure of which is hereby incorporated by reference for all purposes.

### FIELD

This invention generally relates to Cardio Pulmonary Resuscitation, and more particularly, to using a phonocardiogram to assess Cardio Pulmonary Resuscitation effectiveness.

### BACKGROUND

In humans, the heart beats to sustain life. In normal operation, it pumps blood through the various parts of the body. More particularly, the various chambers of the heart contract and expand in a periodic and coordinated fashion, which causes the blood to be pumped regularly. More specifically, the right atrium sends deoxygenated blood into the right ventricle. The right ventricle pumps the blood to the lungs, where it becomes oxygenated, and from where it returns to the left atrium. The left atrium pumps the oxygenated blood to the left ventricle. The left ventricle, then, expels the blood, forcing it to circulate to the various parts of the body.

The heart chambers pump because of the heart's electrical control system. More particularly, the sinoatrial (SA) node generates an electrical impulse, which generates further electrical signals. These further signals cause the above-described contractions of the various chambers in the heart, in the right sequence. The electrical pattern created by the sinoatrial (SA) node is called a sinus rhythm.

Sometimes, however, the electrical control system of the heart malfunctions, which can cause the heart to beat irregularly, or not at all. The cardiac rhythm is then generally called an arrhythmia, and some of it may be caused by electrical activity from locations in the heart other than the SA node. Some types of arrhythmias may result in inadequate blood flow, thus reducing the amount of blood pumped to the various parts of the body. Some arrhythmias may even result in a Sudden Cardiac Arrest (SCA). In a SCA, the heart fails to pump blood effectively, and death can occur. In fact, it is estimated that SCA results in more than 250,000 deaths per year in the United States alone. Further, a SCA may result from a condition other than an arrhythmia.

One type of arrhythmia associated with SCA is known as Ventricular Fibrillation (VF). VF is a type of malfunction where the ventricles make rapid, uncoordinated movements, instead of the normal contractions. When that happens, the heart does not pump enough blood. The person's condition will deteriorate rapidly and, if not reversed in time, they will die soon, e.g. within ten minutes.

Ventricular Fibrillation can often be reversed using a life-saving device called a defibrillator. A defibrillator, if applied properly, can administer an electrical shock to the heart. The shock may terminate the VF, thus giving the heart the opportunity to resume pumping blood. If VF is not terminated, the shock may be repeated, often at escalating energies.

A challenge with defibrillation is that the electrical shock must be administered very soon after the onset of VF. There is not much time: the survival rate of persons suffering from VF decreases by about 10% for each minute the administration of a defibrillation shock is delayed. After about 10 minutes the rate of survival for SCA victims averages less than 2%.

The challenge of defibrillating early after the onset of VF is being met in a number of ways. First, for some people who are considered to be at a higher risk of VF or other heart arrhythmias, an Implantable Cardioverter Defibrillator (ICD) can be implanted surgically. An ICD can monitor the person's heart, and administer an electrical shock as needed. As such, an ICD reduces the need to have the higher-risk person be monitored constantly by medical personnel.

Regardless, VF can occur unpredictably, even to a person who is not considered at risk. As such, VF can be experienced by many people who lack the benefit of ICD therapy. When VF occurs to a person who does not have an ICD, they collapse, because blood flow has stopped. They should receive therapy quickly.

For a VF victim without an ICD, a different type of defibrillator can be used, which is called an external defibrillator. External defibrillators have been made portable, so they can be brought to a potential VF victim quickly enough to revive them.

During VF, the person's condition deteriorates, because the blood is not flowing to the brain, heart, lungs, and other organs. Blood flow must be restored, if resuscitation attempts are to be successful.

Cardiopulmonary Resuscitation (CPR) is one method of forcing blood flow in a person experiencing cardiac arrest. In addition, CPR is the primary recommended treatment for some patients with some kinds of non-VF cardiac arrest, such as asystole and pulseless electrical activity (PEA). CPR is a combination of techniques that include chest compressions to force blood circulation, and rescue breathing to force respiration.

Properly administered CPR provides oxygenated blood to critical organs of a person in cardiac arrest, thereby minimizing the deterioration that would otherwise occur. As such, CPR can be beneficial for persons experiencing VF, because it slows the deterioration that would otherwise occur while a defibrillator is being retrieved. Indeed, for patients with an extended down-time, survival rates are higher if CPR is administered prior to defibrillation.

It is desirable to increase the effectiveness of CPR.

### BRIEF DESCRIPTION OF THE DRAWINGS

FIG. 1 is a diagram of a scene where an external defibrillator is used to save the life of a person according to embodiments.

FIG. 2 is a table listing two main types of the external defibrillator shown in FIG. 1, and who they might be used by.

FIG. 3 is a diagram illustrating components of an external defibrillator, such as the one shown in FIG. 1, which is made according to embodiments.

FIG. 4 is a conceptual diagram of a system according to embodiments.

FIG. 5 is a graph illustrating how a gating signal is generated according to embodiments.

FIGS. 6A, 6B, and 6C are graphs illustrating how the gating signal of FIG. 5 can relate to different patterns of chest compression depths, according to embodiments.

FIG. 7 is a diagram of various signal or sound generating devices according to embodiments of the system of FIG. 4.

FIG. 8 is a diagram illustrating a detailed example of a signal generating device of FIG. 7, according to embodiments.

FIG. 9 is a diagram illustrating a detailed example of a sound generating device of FIG. 7, according to embodiments.

FIG. 10 is a functional block diagram illustrating components of a device for analyzing phonocardiograms according to embodiments.

FIG. 11 is an example flow diagram for illustrating methods according to embodiments.

FIG. 12A is a graph of a sound recording in embodiments.

FIG. 12B is a timing graph of a gating output derived from a gating signal that is used to gate some portions of the sound recording of FIG. 12A but not other portions, according to embodiments.

FIG. 12C is the graph of FIG. 12A, after it has been gated by the gating output of FIG. 12B, which can be used to determine the effectiveness of the chest compressions, according to embodiments.

### DETAILED DESCRIPTION

The present description is about systems for providing status information about resuscitation efforts of a person receiving chest compressions as a part of Cardiopulmonary Resuscitation (CPR). Such systems may include devices, control systems, software and methods, as well as other embodiments. Embodiments are now described in more detail.

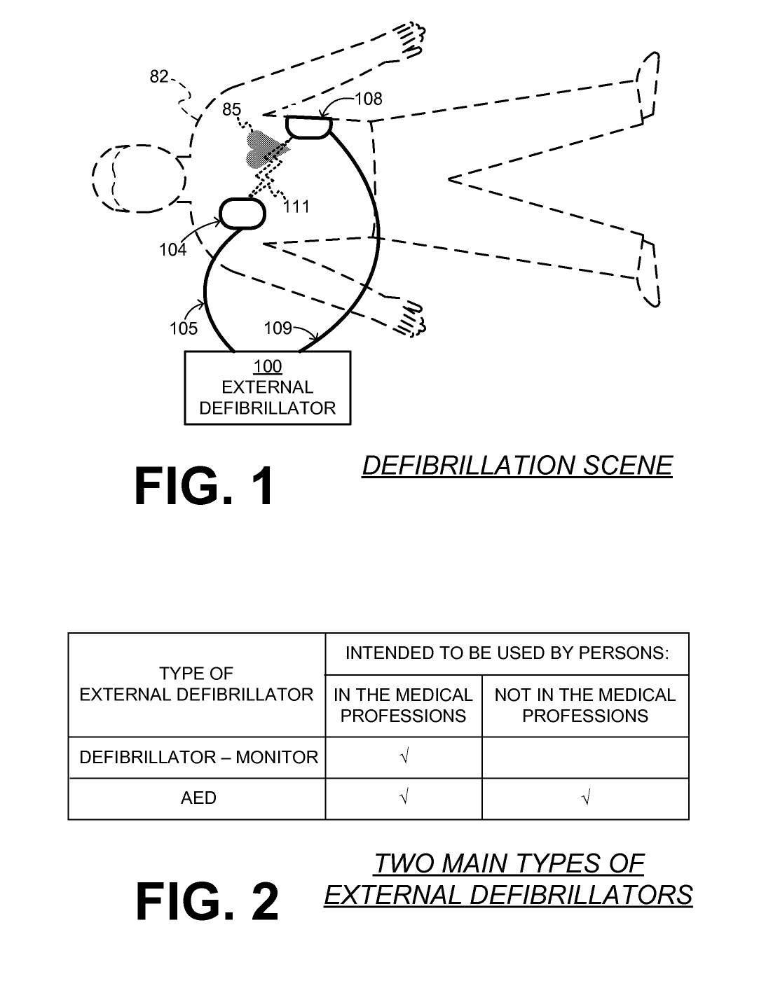

FIG. 1 is a diagram of a defibrillation scene. A person 82 is lying on their back. Person 82 could be a patient in a hospital, or someone found unconscious, and then turned to be on their back. Person 82 is experiencing a condition in their heart 85, which could be Ventricular Fibrillation (VF).

A portable external defibrillator 100 has been brought close to person 82. At least two defibrillation electrodes 104, 108 are usually provided with external defibrillator 100, and are sometimes called electrodes 104, 108. Electrodes 104, 108 are coupled with external defibrillator 100 via respective electrode leads 105, 109. A rescuer (not shown) has attached electrodes 104, 108 to the skin of person 82. Defibrillator 100 is administering, via electrodes 104, 108, a brief, strong electric pulse 111 through the body of person 82. Pulse 111, also known as a defibrillation shock, goes also through heart 85, in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing two main types of external defibrillators, and who they are primarily intended to be used by. A first type of defibrillator 100 is generally called a defibrillator-monitor, because it is typically formed as a unit with a patient monitor. A defibrillator-monitor is sometimes called monitor-defibrillator. A defibrillator-monitor is intended to be used by persons in the medical professions, such as doctors, nurses, paramedics, emergency medical technicians, etc. Such a defibrillator-monitor is intended to be used in a pre-hospital or hospital scenario.

As a defibrillator, the device can be one of different varieties, or even versatile enough to be able to switch among different modes that individually correspond to the varieties. One variety is that of an automated defibrillator, which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological signals of a person in an emergency scenario. For example, these signals can include a person's full ECG (electrocardiogram) signals. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These patient signals can be further stored and/or transmitted as patient data.

A second type of external defibrillator 100 is generally called an AED, which stands for “Automated External Defibrillator”. An AED typically makes the shock/no shock determination by itself, automatically. Indeed, it can sense enough physiological conditions of the person 82 via only the shown defibrillation electrodes 104, 108 of FIG. 1. In its present embodiments, an AED can either administer the shock automatically, or instruct the user to do so, e.g. by pushing a button. Being of a much simpler construction, an AED typically costs much less than a defibrillator-monitor. As such, it makes sense for a hospital, for example, to deploy AEDs at its various floors, in case the more expensive defibrillator-monitor is at an Intensive Care Unit, and so on.

AEDs, however, can also be used by people who are not in the medical profession. More particularly, an AED can be used by many professional first responders, such as policemen, firemen, etc. Even a person with only first-aid training can use one. And AEDs increasingly can supply instructions to whoever is using them.

AEDs are thus particularly useful, because it is so critical to respond quickly, when a person suffers from VF. Indeed, the people who will first reach the VF sufferer may not be in the medical professions.

Increasing awareness has resulted in AEDs being deployed in public or semi-public spaces, so that even a member of the public can use one, if they have obtained first aid and CPR/AED training on their own initiative. This way, defibrillation can be administered soon enough after the onset of VF, to hopefully be effective in rescuing the person.

There are additional types of external defibrillators, which are not listed in FIG. 2. For example, a hybrid defibrillator can have aspects of an AED, and also of a defibrillator-monitor. A usual such aspect is additional ECG monitoring capability.

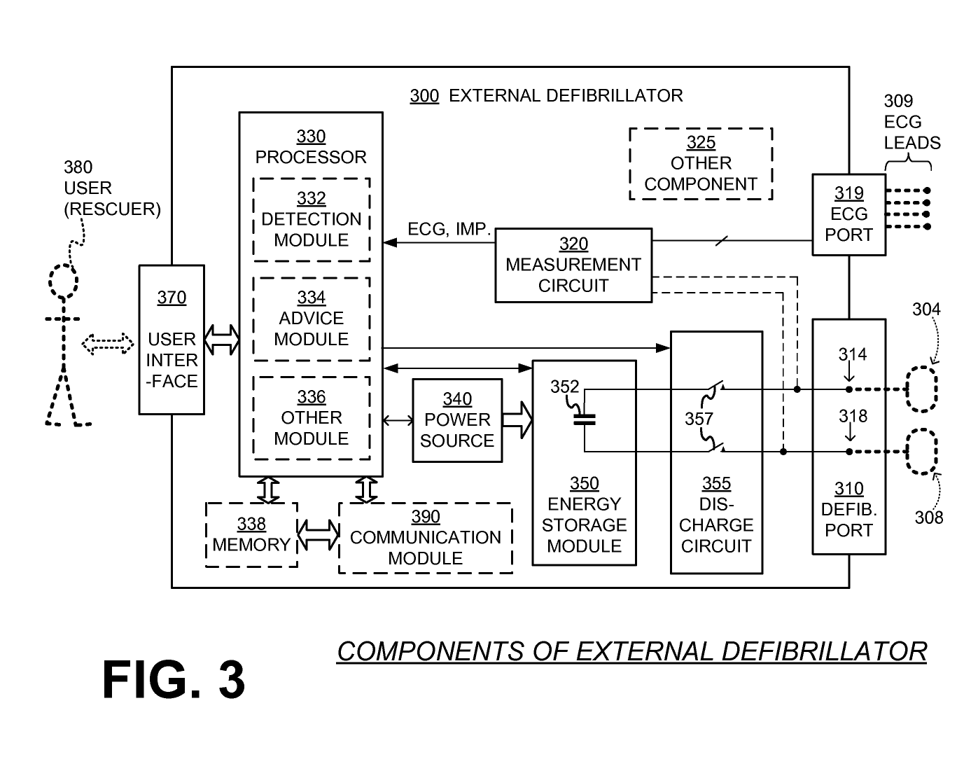

FIG. 3 is a diagram showing components of an external defibrillator 300 made according to embodiments. These components can be, for example, in external defibrillator 100 of FIG. 1.

External defibrillator 300 is intended for use by a user 380, who would be the rescuer. Defibrillator 300 typically includes a defibrillation port 310, such as a socket. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding via electrodes to person 82 an electrical charge that has been stored in defibrillator 300, as will be seen later in this document.

If defibrillator 300 is actually a defibrillator-monitor, as was described with reference to FIG. 2, then it will typically also have an ECG port 319, for plugging in ECG leads 309. ECG leads 309 can help sense an ECG signal, e.g. a 12-lead signal, or from a different number of leads. Moreover, a defibrillator-monitor could have additional ports (not shown), and an other component 325 for the above described additional features, such as patient signals.

Defibrillator 300 also includes a measurement circuit 320. Measurement circuit 320 receives physiological signals from ECG port 319, and also from other ports, if provided. These physiological signals are sensed, and information about them is rendered by circuit 320 as data, or other signals, etc.

If defibrillator 300 is actually an AED, it may lack ECG port 319. Measurement circuit 320 can obtain physiological signals through nodes 314, 318 instead, when defibrillation electrodes 304, 308 are attached to person 82. In these cases, a person's ECG signal can be sensed as a voltage difference between electrodes 304, 308. Plus, impedance between electrodes 304, 308 can be sensed for detecting, among other things, whether these electrodes 304, 308 have been inadvertently disconnected from the person.

Defibrillator 300 also includes a processor 330. Processor 330 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. Advice module 334 can include a Shock Advisory Algorithm, implement decision rules, and so on. The advice can be to shock, to not shock, to administer other forms of therapy, and so on. If the advice is to shock, some external defibrillator embodiments merely report that to the user, and prompt them to do it. Other embodiments further execute the advice, by administering the shock. If the advice is to administer CPR, defibrillator 300 may further issue prompts for it, and so on.

Processor 330 can include additional modules, such as module 336, for other functions. In addition, if other component 325 is indeed provided, it may be operated in part by processor 330, etc.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. The programs can be operational for the inherent needs of processor 330, and can also include protocols and ways that decisions can be made by advice module 334. In addition, memory 338 can store prompts for user 380, etc. Moreover, memory 338 can store patient data.

Defibrillator 300 may also include a power source 340. To enable portability of defibrillator 300, power source 340 typically includes a battery. Such a battery is typically implemented as a battery pack, which can be rechargeable or not. Sometimes, a combination is used, of rechargeable and non-rechargeable battery packs. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, and so on.

Defibrillator 300 moreover includes a discharge circuit 355. Circuit 355 can be controlled to permit the energy stored in module 350 to be discharged to nodes 314, 318, and thus also to defibrillation electrodes 304, 308. Circuit 355 can include one or more switches 357. Those can be made in a number of ways, such as by an H-bridge, and so on.

Defibrillator 300 further includes a user interface 370 for user 380. User interface 370 can be made in any number of ways. For example, interface 370 may include a screen, to display what is detected and measured, provide visual feedback to the rescuer for their resuscitation attempts, and so on. Interface 370 may also include a speaker, to issue voice prompts, etc. Interface 370 may additionally include various controls, such as pushbuttons, keyboards, and so on. In addition, discharge circuit 355 can be controlled by processor 330, or directly by user 380 via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.

Additional components can include those for detecting the effectiveness of CPR that a rescuer might be delivering. In embodiments, a Phonocardiogram is used. A microphone or other sound sensor is used to generate a sound track, i.e. a sound input, from sounds sensed from the patient's body. The microphone or other sound sensor can be placed in contact with the body, perhaps integrated with the electrodes, etc. Sounds are then analyzed to determine the effectiveness of the resuscitation efforts. In particular, sounds can be analyzed to determine action of the heart valves, i.e., whether and to what extent they are opening, closing, and how much blood is moving past them during the CPR.

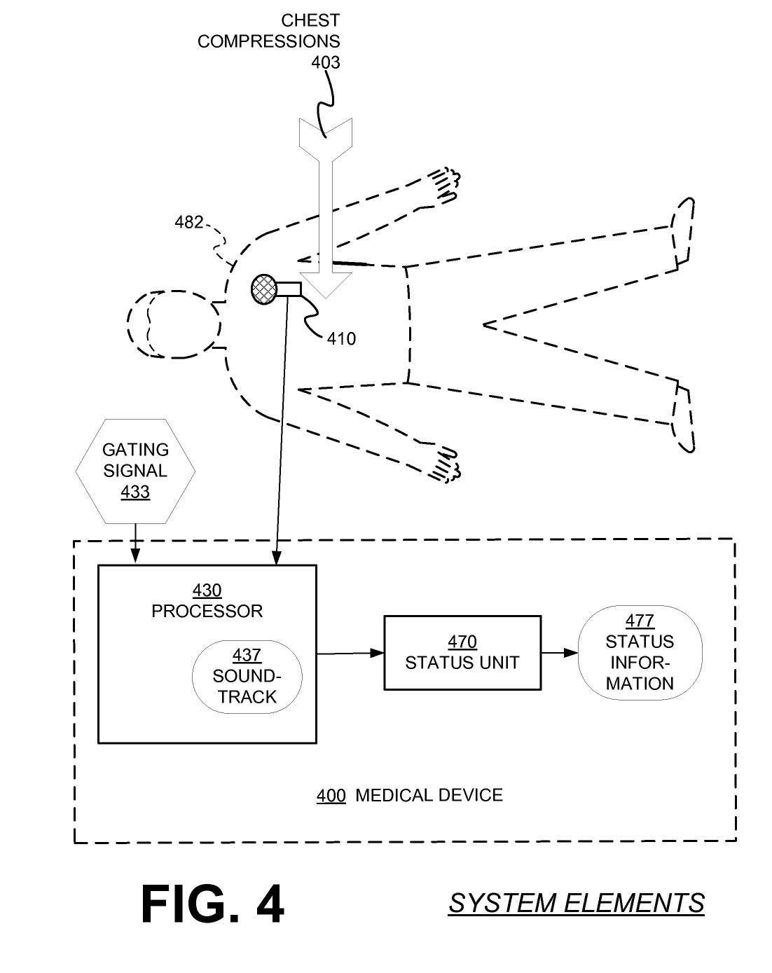

FIG. 4 is a conceptual diagram of an example system for providing status information about resuscitation efforts of a person receiving chest compressions as part of CPR. In FIG. 4 a patient 482 is receiving, has received, or will receive chest compressions 403 as part of CPR. A microphone 410 for sampling sounds within a body of the person to generate a soundtrack is placed on, near, or even within the body cavity of the patient 482. In the embodiment of FIG. 4, the microphone 410 has been placed near the heart. In other embodiments the microphone 410 may be placed on a peripheral artery of the patient, rather than near the heart. In some embodiments the microphone 410 may be integrated with a blood pressure cuff. With reference back to FIG. 3, although not as illustrated in FIG. 4, the microphone 410 may be attached to or integrated into electrodes 304, 308, which, in turn, may be adhered to the patient 482.

The microphone 410, or other signal generating instrument, generates a soundtrack 437 for a medical device 400 as shown in FIG. 4. By “soundtrack” in this document, it is meant sound recording. In some embodiments the soundtrack may be generated by or supplemented with output from a Doppler ultrasound (not illustrated), which may generate a signal based on the movement of blood within the patient. In addition to the soundtrack 437, a gating signal 433, which is presumed to be correlated to the chest compressions, is also received by a processor within the medical device. The processor determines a cardiovascular effect of the CPR compressions using portions of the soundtrack gated by the gating signal to determine action of the heart, or cardiovascular effect, of the patient 482 during or in close association with the CPR the patient is receiving, as described in detail below. The processor then determines status information 477 from the cardiovascular effect. A status unit 470 is coupled to the processor and structured to output the status information.

In some embodiments the cardiovascular effect determined by the processor is a heart valve closure of the person receiving chest compressions. This can be performed in a number of ways.

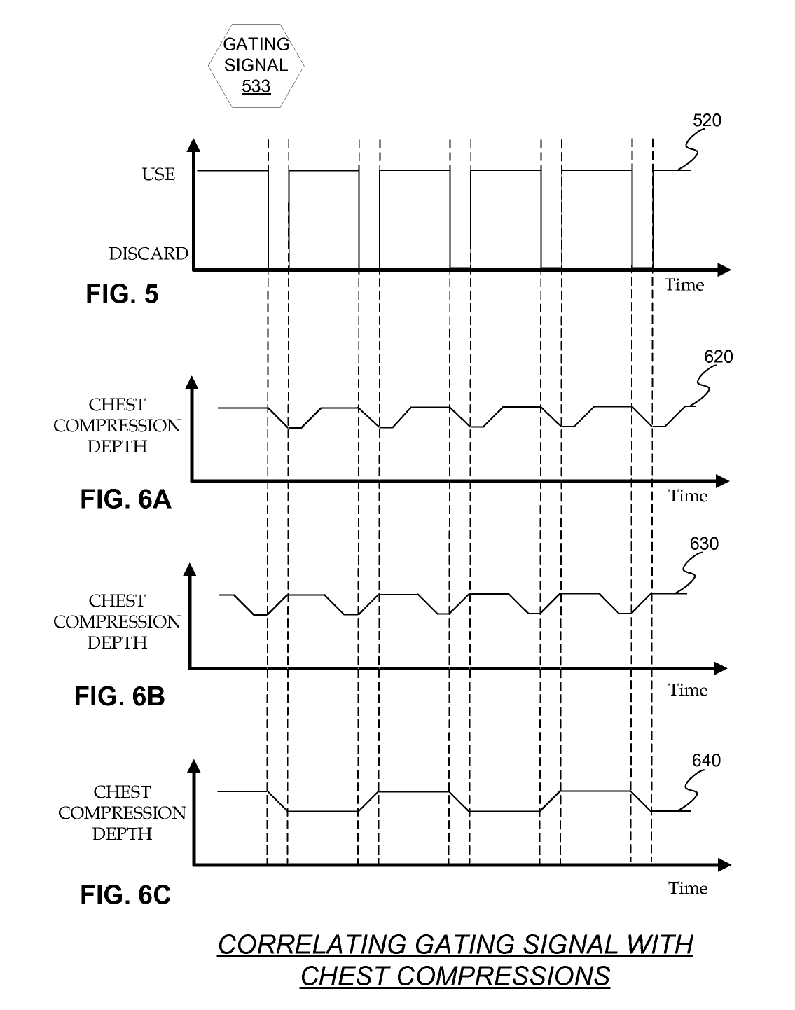

FIG. 5 is a time plot that illustrates how a gating signal can be generated according to embodiments. A signal 520 has two binary states that are determined by a gating signal generator, described in detail below. In general, portions of the soundtrack generated by the microphone 410 are discarded when the gating signal is in a “discard” state, and portions of the soundtrack generated by the microphone 410 are used when the gating signal is in a “use” state. Analysis of the soundtrack allows the system to determine the effectiveness of the CPR, which may then be communicated to the rescuer. There are several ways or methods to determine how to generate the gating signal, also described below. Generally, the discarded portions of the soundtrack relate to times during which the chest compressions are occurring, which is necessarily “noisy,” and masks the sounds of the cardiovascular effect sampled by the microphone 410.

FIGS. 6A-6C illustrate how the gating signal 533 is generated from a signal related to chest compressions. FIG. 6A illustrates a signal 620 that correlates to a depth of a chest compression that the patient is receiving during CPR. The signal 620 is in a HIGH state when the chest is in an uncompressed, or natural, state, and the signal is in a LOW state when the chest is in a fully compressed state. A time period between the transition from the HIGH state to the LOW state correlates to the DISCARD state of the gating signal 533, illustrated in FIG. 5. Similarly, the DISCARD state of the gating signal 533 translates to the transition time from the LOW state to the HIGH state of the signal 630 of FIG. 6B. As illustrated in FIG. 6C, the DISCARD state of the gating signal 533 corresponds to both the transitions from LOW to HIGH and from HIGH to LOW of the signal 640. Of course, these are but only some examples of how the gating signal 533 can be generated.

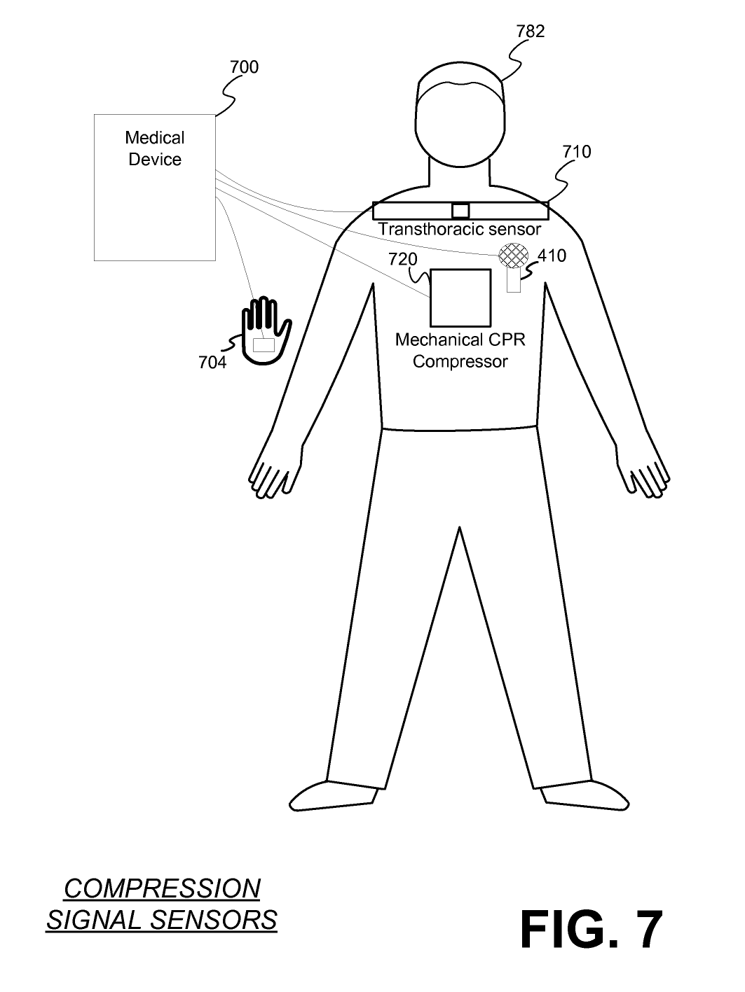

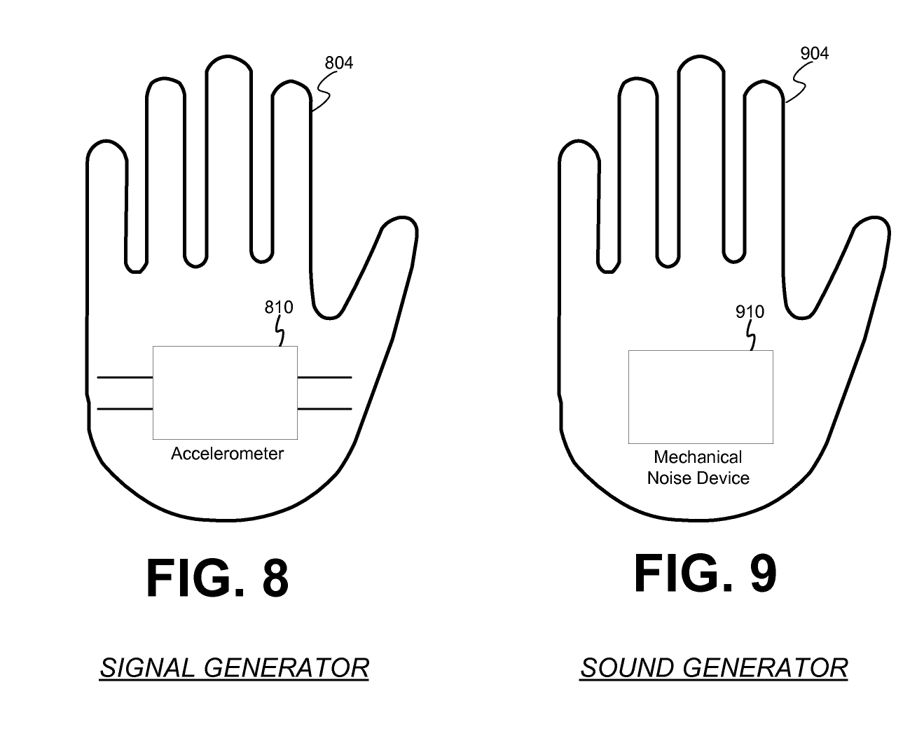

FIG. 7 illustrates how a signal can be generated for analysis in creating the gating signal 533. For instance a transthoracic sensor 710 detects chest movement by measuring changes to a carrier signal during compressions, due to a change in impedance of the carrier signal during the compressions. The transthoracic sensor 710 may be a stand-alone sensor, or may be integrated into the defibrillation electrodes 304, 308 of FIG. 3. In other embodiments the sensor 710 may be integrated into a separate monitoring pad. In another embodiment a signal may be generated by a mechanical CPR compressor 720. In such an embodiment the mechanical compressor 720 physically compresses the chest for CPR, and generates a signal that relates to the position of the chest during such compressions. In yet another embodiment, sensors or other devices may be attached to a hand 704 of the rescuer. Examples of devices are illustrated in FIGS. 8 and 9.

FIG. 8 illustrates an accelerometer 810 mounted to a hand 804 of the rescuer. In one embodiment the accelerometer 810 is attached by a strap or band, while in other embodiments the accelerometer may be attached by adhesive or through other means. As is known in the art, an accelerometer detects motion, specifically acceleration, and generates a signal related to the sensed motion. The generated signal generally includes both direction and magnitude which, combined with a time signal, may be translated to velocity and distance. A final output of the signal may be similar to or may be modified to create the signals 620, 630, and 640 of FIGS. 6A-6C.

FIG. 9 illustrates a sound generator 910 also attached to a hand 904 of a rescuer. The sound generator may produce an audible “click” or other noise when pressed by the rescuer. The sound generator 910 may in fact be placed below the hand 904, between the hand and the chest of the person receiving CPR. The microphone 410 (FIG. 4) records the sound produced by the sound generator 910, which may be used to mark the soundtrack when a chest compression is occurring.

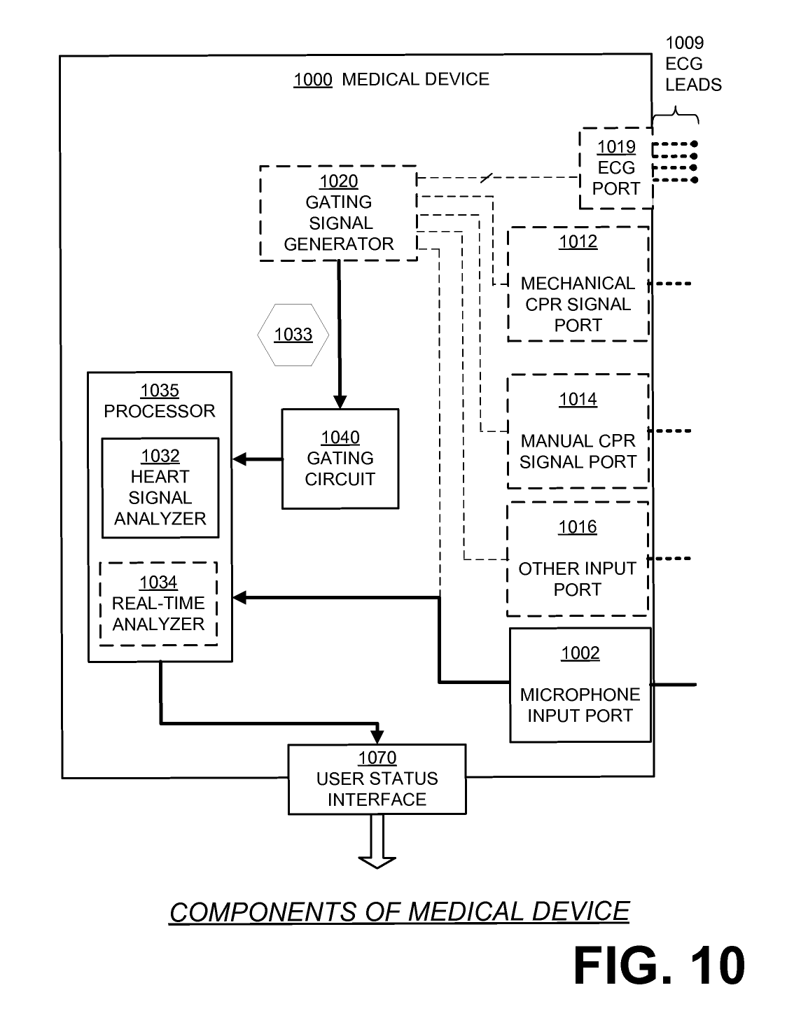

The sounds or signals generated by any of the methods of FIGS. 7-9 may act as inputs to a medical device, for example the medical device 1000 of FIG. 10. In FIG. 10, each signal or sound generator is received through a respective port, 1012, 1014, and/or 1016. Additionally the soundtrack from the microphone 410 is received through the input port 1002. The soundtrack is presented directly to a processor 1035, while the signals from the ports 1012, 1014, and 1016, if present, are passed to a gating signal generator 1020. The gating signal generator receives the input from the one or more ports and generates a gating signal 1033, which in turn is passed to a gating circuit 1040. The gating signal 1033 may appear in a similar form to that illustrated in FIG. 5. In some embodiments the gating signal 1033 may be determined by analysis of the soundtrack only, without any signals from the other signal ports 1012, 1014, and 1016. In a software embodiment, where the processor is executing instructions running on a special purpose or general purpose processor, the gating signal 1033 may be a software value. In other embodiments the gating signal 1033 may be generated by an automated chest compression machine, passed to the medical device 1000 through the mechanical CPR signal port 1012. In yet other embodiments, particular sounds may be generated and injected into the patient's body, or near the patient, where they are detected by the microphone 410 (FIG. 4), or a separate microphone (not illustrated). These generated sounds may be periodic or substantially periodic, and may be generated by the mechanical CPR compressor 720 of FIG. 7.

The processor 1055 then analyzes the cardiovascular effect, such as heart valve closure or blood flowing through the body, from the soundtrack sensed by the microphone 410 (FIG. 4) to determine a physiological effect that the CPR is having on the patient. In some embodiments the processor 1035 determines a time interval from a compression action of the compressions until the heart valve closure. The processor 1035 may be the same or similar to the processor 330 of FIG. 3. The processor 1035 generates a signal for a user status interface 1070, which may be used to generate an output for the rescuer so that the rescuer may gauge how well the CPR is working. In some embodiments the user status interface 1070 may generate sounds, lights, or audible prompts, etc., for the rescuer, to provide CPR coaching. The status information produced by the status interface 1070 may, in fact, reflect a determination that the compressions are not effective for the patient.

In some embodiments the cardiovascular effect determined by the processor 1035 may be that the patient has had a Return of Spontaneous Circulation (ROSC). This could be determined by concluding that, based on the soundtrack, the heart valves are operating on their own and/or blood is flowing through the body for a reason other than the CPR.

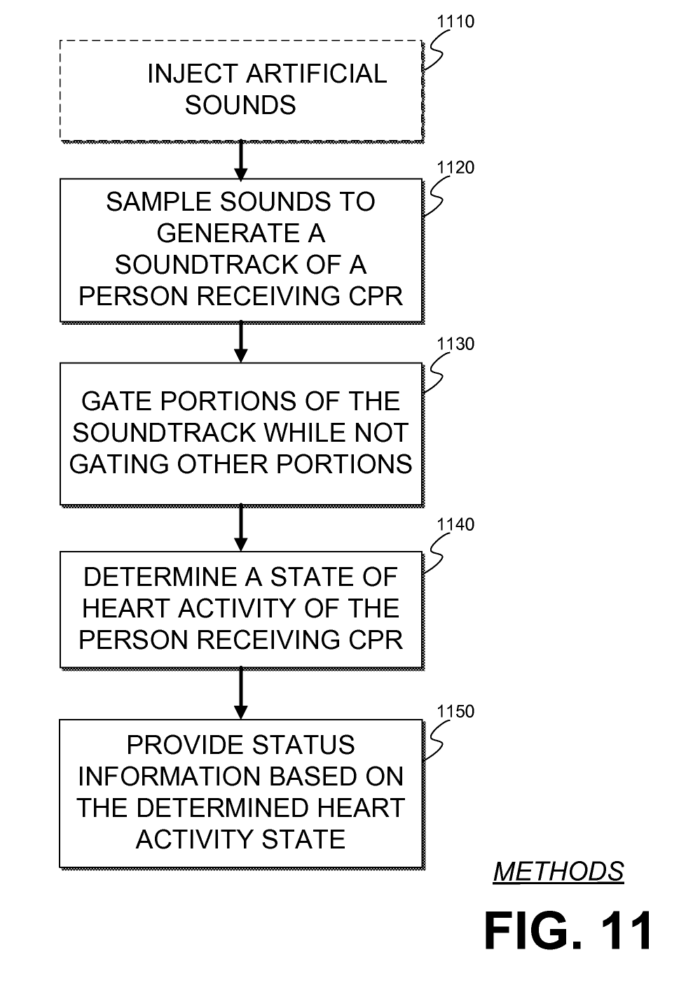

FIG. 11 is an example flow diagram for illustrating methods according to embodiments. The method of the flow diagram of FIG. 11 may also be implemented by the medical device 1000 of FIG. 10, the external defibrillator 300 of FIG. 3, by a processor executing instructions, or in other manners according to embodiments. According to an optional operation 1110, sounds are injected into or about a patient receiving CPR, such as the patient 482 of FIG. 4. According to a next operation 1120, a microphone or other device samples sounds of the patient and/or the environment around the patient to generate a soundtrack of a person receiving CPR. According to a next operation 1130, the soundtrack generated in operation 1120 is “gated,” i.e. parsed by a gating signal. According to a next operation 1140 a state of heart activity of the person receiving CPR is determined from the gated soundtrack. According to a next operation 1150 status of the determined activity from the operation 1140 is provided to the rescuer or another person associated with the rescue.

According to some embodiments, determining the cardiovascular effect comprises determining a heart valve closure of the person receiving chest compressions. In some embodiments the cardiovascular effect determined is that the patient has returned to spontaneous circulation. According to other embodiments, another optional step may include determining a time interval from a compression action of the compressions until the heart valve closure.

Other embodiments may include a determination that the chest compressions are not effective for the person receiving chest compressions for a physiological reason, such as massive pulmonary embolism.

As described above, the gating signal used in the operation 1130 of FIG. 11 correlates with a compressing action of the compressions. In other embodiments the gating signal correlates with a releasing action of the compressions. In yet further embodiments the gating signal correlates with both the compression and releasing actions. A signal that reports the depth of the chest compressions may also be used to generate the gating signal, or for other purposes, such as to provide feedback to the rescuer that compressions should be deeper or more shallow for maximum CPR effect.

In some embodiments the gating signal may be an electrical signal or a software value. The gating signal may be generated by an automated chest compression machine. In some embodiments an artificial sound, such as a periodic or substantially periodic signal may be produced and projected into the patient or environment of the patient. The artificial sound may then be used to generate the gating signal. In other embodiments the automated chest compression machine generates the artificial sound. The artificial sound may be sensed by a microphone placed on, in, or about the patient, or the microphone may be integrated into a monitoring or defibrillation pad.

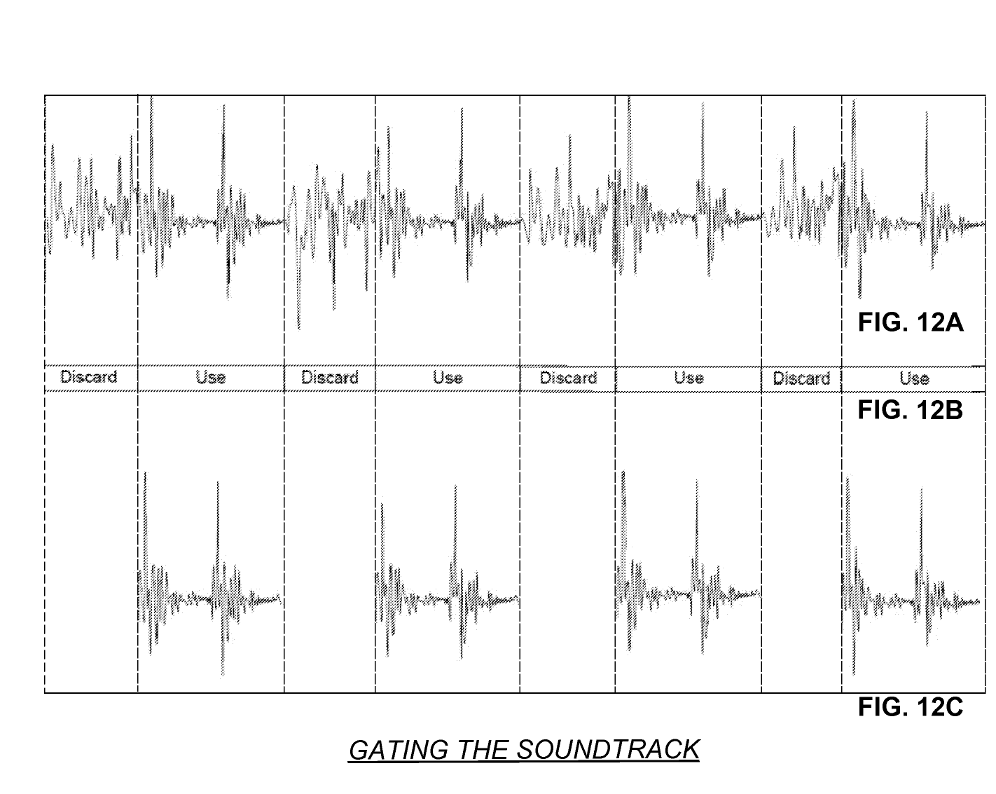

FIGS. 12A, 12B, and 12C are diagrams that illustrate operation of the above-described system according to embodiments. FIG. 12 illustrates a soundtrack generated by a microphone 410 (FIG. 4) or other apparatus placed in, on, or near a patient receiving CPR. As illustrated the signal may be relatively noisy. FIG. 12 B is a state diagram illustrating a state of the gating signal, such as the gating signal 533 of FIG. 5. As described above, the DISCARD state relates to periods where the chest is being compressed, released, or both. FIG. 12 C illustrates the soundtrack signal 12A parsed according to the state of the gating signal illustrated in FIG. 12 B. The processor 1035 of FIG. 10 then uses the parsed soundtrack, such as that illustrated in FIG. 12 C to determine the cardiovascular effect that the compressions are having on the patient receiving CPR.

In this description, numerous details have been set forth in order to provide a thorough understanding. In other instances, well-known features have not been described in detail in order to not obscure unnecessarily the description.

A person skilled in the art will be able to practice the present invention in view of this description, which is to be taken as a whole. The specific embodiments as disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems.

The following claims define certain combinations and subcombinations of elements, features, steps, and/or functions, which are regarded as novel and non-obvious. Additional claims for other combinations and subcombinations may be presented in this or a related document.

What is claimed is:

1. A system for providing status information about resuscitation efforts of a person receiving chest compressions as part of Cardiopulmonary Resuscitation (CPR), comprising:
a microphone for sampling sounds within a body of the person to generate a soundtrack;
a processor structured to determine a cardiovascular effect of the compressions from gated portions of the soundtrack but not from other portions, the gated portions and the other portions of the soundtrack determined by a gating signal that is presumed to be correlated with the chest compressions, the processor further structured to determine the status information from the cardiovascular effect; and
a status unit coupled to the processor and structured to output the status information.

2. The system of claim 1, in which the cardiovascular effect is a heart valve closure of the person receiving chest compressions.

3. The system of claim 2, in which the processor is further structured to determine a time interval from a compression action of the compressions until the heart valve closure.

4. The system of claim 1, in which the cardiovascular effect is a Return of Spontaneous Circulation (ROSC) of the person receiving chest compressions.

5. The system of claim 1, in which the status information is a determination that the chest compressions are not effective for the person receiving chest compressions for a physiological reason.

6. The system of claim 1, in which the gating signal correlates with a compressing action of the compressions.

7. The system of claim 6, in which the gating signal correlates with a releasing action between two compressing actions.

8. The system of claim 1, in which the gating signal is one of an electrical signal and a software value.

9. The system of claim 1, in which the gating signal is generated based on the soundtrack.

10. The system of claim 1, in which the gating signal is received as issued by an automated chest compression machine.

11. The system of claim 1, further comprising:
a signal generator for producing an artificial sound; and
a gating signal generator for using the artificial sound as an input and structured to generate the gating signal in response thereto.

12. The system of claim 11, in which producing the artificial sound comprises injecting an artificial sound to the person's body.

13. The system of claim 11, in which the signal generator is coupled to a hand of a rescuer providing the chest compressions.

14. The system of claim 11, in which the artificial sound is substantially periodic.

15. The system of claim 11, in which the artificial sound is correlated with an action of a mechanical chest compression device that provides the chest compressions.

16. The system of claim 1, further comprising:
a sensor for generating a compression input related to the chest compressions; and
a gating signal generator coupled to the sensor and structured to generate the gating signal from the compression input.

17. The system of claim 16, in which the sensor is a transthoracic impedance sensor.

18. The system of claim 16, in which the sensor senses a depth of the chest compressions.

19. The system of claim 1, further comprising:
a capacitor for storing a charge with which to defibrillate the person; and
in which the status unit is further adapted to issue instructions for the defibrillation.

20. The system of claim 1, in which the microphone is attached or integrated into a pad.

21. The system of claim 20, in which the pad is a monitoring pad or a defibrillation pad.

22. The system of claim 1, further comprising a soundtrack analyzer for providing CPR coaching.

23. The system of claim 1, further comprising a second microphone structured to sample environmental sounds.

24. A method for providing status information about resuscitation efforts of a person receiving chest compressions, comprising:
sampling sounds of the body of the person to generate a soundtrack;
gating some portions of the soundtrack but not other portions of the soundtrack as determined by a gating signal that is presumed to correlate to the chest compressions;
determining a cardiovascular effect of the compressions from the gated portions of the soundtrack but not other portions; and
providing the status information based on the determined cardiovascular effect.

25. The method of claim 24, in which determining the cardiovascular effect comprises determining a heart valve closure of the person receiving chest compressions.

26. The method of claim 24, further comprising determining a time interval from a compression action of the compressions until the heart valve closure.

27. The method of claim 26, in which determining the cardiovascular effect comprises determining a Return of Spontaneous Circulation (ROSC) of the person receiving chest compressions.

28. The method of claim 24, in which the status information is a determination that the chest compressions are not effective for the person receiving chest compressions for a physiological reason.

29. The method of claim 24, in which the gating signal correlates with a compressing action of the compressions.

30. The method of claim 29, in which the gating signal correlates with a releasing action between two compressing actions.

31. The method of claim 24, in which the gating signal can be an electrical signal or a software value.

32. The method of claim 24, further comprising providing an indicator of the determined cardiovascular effect to a rescuer providing the chest compressions.

33. The method of claim 24, further comprising generating a gating signal presumed to be correlated with the chest compressions.

34. The method of claim 24, further comprising receiving the gating signal as issued from an automated chest compression machine.

35. The method of claim 24, further comprising:
producing an artificial sound; and
generating the gating signal based on the artificial sound.

36. The method of claim 35, in which producing an artificial sound comprises injecting the artificial sound to the person's body.

37. The method of claim 35, in which the artificial sound is substantially periodic.

38. The method of claim 35, in which the artificial sound is correlated with an action of a mechanical chest compression device that provides the chest compressions.

39. The method of claim 35, further comprising measuring a depth of the chest compressions.

40. The method of claim 24, in which the sounds are sampled by microphone.

41. The method of claim 40 in which the microphone is attached or integrated into a pad.

42. The method of claim 41, in which the pad is a monitoring pad or a defibrillation pad.

43. The method of claim 40, further comprising sampling environmental sounds.

44. The method of claim 24, further comprising analyzing the soundtrack; and
providing Cardiopulmonary Resuscitation (CPR) coaching.
